(19) United States
(12) Patent Application Publication
(10) Pub. No.: US 2019/0357775 A1
(43) Pub. Date: Nov. 28, 2019

(54) BIOMETRIC APPARATUS

(71) Applicant: SEIKO EPSON CORPORATION, Tokyo (JP)

(72) Inventor: Satoshi Takenaka, Chino-shi (JP)

(73) Assignee: SEIKO EPSON CORPORATION, Tokyo (JP)

(21) Appl. No.: 16/420,939

(22) Filed: May 23, 2019

(30) Foreign Application Priority Data
May 24, 2018 (JP) 2018-099411

(51) Int. Cl. A61B 5/022; A61B 5/00

(52) U.S. Cl. CPC A61B 5/022; A61B 2560/0462; A61B 5/681

(57) ABSTRACT

There is provided a biometric apparatus that includes a detector generating a detection signal in response to a state of a living body; a mounting unit mounting the detector to the living body, and including a pressure sensing unit made of pressure-sensitive conductive resin and configured to elastically stretch and contract, and a first electrode and a second electrode formed on the pressure sensing unit; a calculation unit calculating an evaluation index in response to a resistance between the first electrode and the second electrode; and an evaluation unit evaluating a mounting state of the detector in response to the evaluation index.

The present application is based on, and claims priority from JP Application Serial Number 2018-099411, filed May 24, 2018, the disclosure of which is hereby incorporated by reference herein in its entirety.

### BACKGROUND

### 1. Technical Field

The present disclosure relates to a technology for analyzing a state of a living body.

### 2. Related Art

Various related technologies have been proposed to measure biometric information such as a pulse rate. JP-A-2006-247133 discloses a wristwatch type biosignal measurement apparatus including a detection unit that generates biosignals in response to a state of a living body, and a belt-shaped mounting unit that mounts the detection unit on the living body. In the technology disclosed in JP-A-2006-247133, the detection unit measures a pressure on a living body using a pressure sensor installed on the mounting unit, and the appropriateness of a measurement environment is evaluated based on a measurement result.

However, in the technology disclosed in JP-A-2006-247133, because a pressure on a living body is measured by the pressure sensor, the evaluation of a measurement environment depends on a state of a local contact between the pressure sensor and a surface of the living body. That is, the technology disclosed in JP-A-2006-247133 has a problem that the mounting state of the detection unit cannot necessarily be appropriately evaluated.

### SUMMARY

A biometric apparatus according to a preferred aspect of the present disclosure includes a detector generating a detection signal in response to a state of a living body; a mounting unit mounting the detector to the living body, and including a pressure sensing unit made of pressure-sensitive conductive resin and configured to elastically stretch and contract, and a first electrode and a second electrode formed in the pressure sensing unit; a calculation unit calculating an evaluation index in response to a resistance between the first electrode and the second electrode; and an evaluation unit evaluating a mounting state of the detector in response to the evaluation index.

### BRIEF DESCRIPTION OF THE DRAWINGS

FIG. 1 is a lateral view of a biometric apparatus according to a first embodiment of the present disclosure.

FIG. 2 is a plan view of the biometric apparatus.

FIG. 3 is a block diagram illustrating a configuration of the biometric apparatus.

FIG. 4 is a block diagram illustrating a functional configuration of the biometric apparatus.

FIG. 5 is a flowchart illustrating a specific sequence of an evaluation process.

FIG. 6 is a plan view of a first electrode and a second electrode in a second embodiment.

FIG. 7 is a lateral view of a biometric apparatus in a third embodiment.

FIG. 8 illustrates a plan view and a lateral view of a first portion of a biometric apparatus in a fourth embodiment.

FIG. 9 is a lateral view of a biometric apparatus in a fifth embodiment.

FIG. 10 is a block diagram illustrating a configuration of a modification example.

### DESCRIPTION OF EXEMPLARY EMBODIMENTS

### First Embodiment

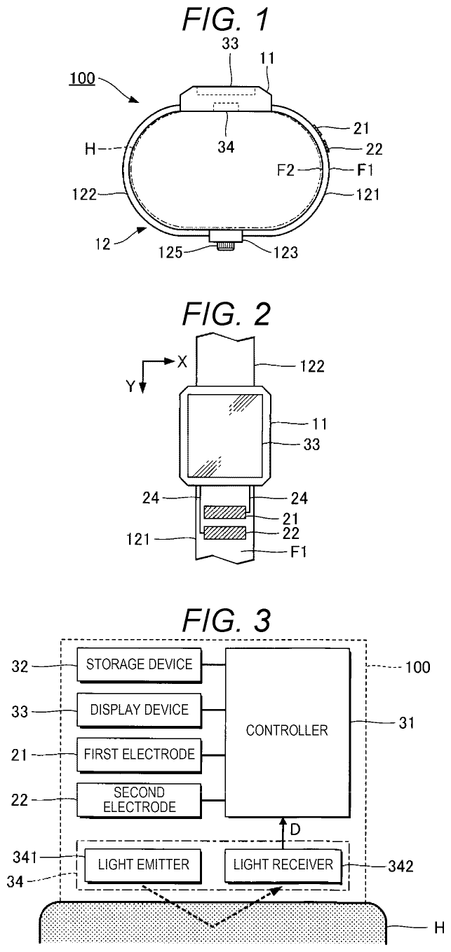

FIG. 1 is a lateral view of a biometric apparatus 100 in a first embodiment of the present disclosure. FIG. 2 is a plan view of the biometric apparatus 100. The biometric apparatus 100 is a measurement device that non-invasively measures biometric information of a subject. The biometric apparatus 100 of the first embodiment is a sphygmograph that measures a pulse rate of the subject as biometric information. The biometric apparatus 100 is mounted on a specific part (hereinafter, referred to as “measurement site”) H of the body of the subject. The measurement site H is a wrist or an upper arm of the subject. The measurement site H is a specific example of a “living body”.

As illustrated in FIG. 1, the biometric apparatus 100 of the first embodiment is a wristwatch type portable device with a housing unit 11 and a mounting unit 12. The housing unit 11 is a hollow structure that accommodates elements of the biometric apparatus 100. The mounting unit 12 is a member that mounts the biometric apparatus 100 on the measurement site H, and includes a first portion 121, a second portion 122, and an adjustment section 123.

Each of the first portion 121 and the second portion 122 is a long planar belt-shaped member that winds around the measurement site H of the subject. A proximal portion of the first portion 121 is coupled to the housing unit 11, and the adjustment section 123 is installed on a distal portion of the first portion 121. A proximal portion of the second portion 122 is coupled to the housing unit 11, and a distal portion of the second portion 122 is detachably coupled to the adjustment section 123. Because the distal portion of the second portion 122 and the distal portion of the first portion 121 are coupled to the adjustment section 123, the biometric apparatus 100 is mounted on the measurement site H in a state where the first portion 121 and the second portion 122 surround the measurement site H.

The adjustment section 123 is a mechanism for adjusting a full length of the first portion 121 and the second portion 122. The adjustment section 123 of the first embodiment includes an operation knob 125 that a user can operate. A user can adjust the full length of the first portion 121 and the second portion 122 by appropriately operating the operation knob 125. That is, a user can arbitrarily adjust the degree of tightening of the mounting unit 12 to the measurement site H.

Each of the first portion 121 and the second portion 122 is made of pressure-sensitive conductive resin, and thus can elastically stretch and contract. The pressure-sensitive conductive resin is a resin material, the resistance of which changes in response to a load. The resistance of the pressure-sensitive conductive resin increases when a tensile load is applied thereto, and the resistance decreases when a compression load is applied thereto. Specifically, the first portion 121 and the second portion 122 is made of the pressure-sensitive conductive resin such as a pressure-sensitive conductive elastomer that is a resin material with multiple conductive particles scattered therein, or magnetic compound fluid (MCF) rubber obtained by mixing magnetic mixed fluid into silicone oil rubber, and curing the mixture in a magnetic field. The first portion 121 of the first embodiment is a specific example of a “pressure sensing unit”. The material of the second portion 122 is not limited to the pressure-sensitive conductive resin.

A first electrode 21 and a second electrode 22 are formed on a surface F1 of the first portion 121, which is on the opposite side of the measurement site H. The surface F1 is an outer peripheral surface of the mounting unit 12. Each of the first electrode 21 and the second electrode 22 is a conductive pattern made of a conductive material, which is formed on the surface F1 of the first portion 121. As illustrated in FIG. 2, each of the first electrode 21 and the second electrode 22 has an elongate shape extending in an X direction. The X direction is a width direction of the first portion 121. The first electrode 21 and the second electrode 22 are arranged with a gap therebetween in a Y direction intersecting the X direction. The Y direction is a longitudinal direction of the first portion 121. The surface F1 is a specific example of a “first surface”.

The resistance of the first portion 121 between the first electrode 21 and the second electrode 22 changes in response to expansion and contraction of the first portion 121. Specifically, the stronger the tightening of the mounting unit 12 to the measurement site H is, the further a tensile load applied to the first portion 121 increases, and thus the resistance between the first electrode 21 and the second electrode 22 increases. That is, the resistance between the first electrode 21 and the second electrode 22 when the tightening of the mounting unit 12 to the measurement site H is strong is greater than the resistance therebetween when the tightening of the mounting unit 12 to the measurement site H is weak. As understood from the description above, the resistance between the first electrode 21 and the second electrode 22 can be used as an index of the degree of tightening of the mounting unit 12 to the measurement site H. The stronger the tightening of the mounting unit 12 is, the further a detector 34 is pressed against a surface of the measurement site H. Therefore, the resistance between the first electrode 21 and the second electrode 22 is preferably used as an index of the mounting state of the detector 34 on the measurement site H. The mounting state of the detector 34 indicates the magnitude of contact pressure applied from the detector 34 to the surface of the measurement site H.

FIG. 3 is an electrical diagram of the biometric apparatus 100. As illustrated in FIG. 3, the biometric apparatus 100 of the first embodiment includes a controller 31; a storage device 32; a display device 33; and the detector 34. The housing unit 11 accommodates the controller 31 and the storage device 32. As illustrated in FIG. 2, each of the first electrode 21 and the second electrode 22 is electrically connected to the controller 31 via a wire 24 formed on the surface F1 of the first portion 121.

In FIG. 3, the display device 33 is installed on a surface of the housing unit 11, which is on the opposite side of the measurement site H. The display device 33 is a liquid crystal display panel, and displays various images containing a measured pulse rate. The detector 34 is installed in the housing unit 11 while facing the measurement site H, and is in close contact with the measurement site H. The mounting unit 12 of the first embodiment is a member that mounts the detector 34 on the measurement site H.

The detector 34 is an optical sensor module that generates a detection signal D in response to a state of the measurement site H. As illustrated in FIG. 3, the detector 34 of the first embodiment includes a light emitter 341 and a light receiver 342. The light emitter 341 is a light source that irradiates the measurement site H with light. A light emitting element such as a light emitting diode (LED) is preferably used as the light emitter 341. In the first embodiment, the light emitter 341 emits near-infrared light having a wavelength in a range from 800 nm to 1,300 nm. Light emitted from the light emitter 341 is not limited to near-infrared light. The light emitter 341 may be a plurality of light emitting elements that emit lights having different wavelengths.

After light incident to the measurement site H from the light emitter 341 is repeatably reflected and scattered inside the measurement site H, the light emits from the measurement site H, and reaches the light receiver 342. The light receiver 342 generates the detection signal D in response to the intensity of light received from the measurement site H. A photoelectrically converted element, for example, a photodiode generating electric charges in response to a received light intensity, is preferably used as the light receiver 342. A light receiving element with a photoelectrically converted layer made of Indium gallium arsenide (InGaAs) and capable of receiving near-infrared light is preferably used as the light receiver 342. The illustration of an A/D converter converting the detection signal D from analog into digital is omitted for illustrative purposes.

Blood vessels at the measurement site H repeatably dilate and constrict in the heartbeat cycle. Light absorptions of blood in a blood vessel differ between dilation and constriction. Therefore, the detection signal D, which the light receiver 342 generates in response to the intensity of light received from the measurement site H, is a pulse wave signal containing a periodically variable component corresponding to a pulsation component (that is, volume pulse wave) of an artery at the measurement site H.

When the tightening of the mounting unit 12 to the measurement site H is excessively strong, a pressure (hereinafter, referred to as “contact pressure”) applied to the measurement site H from the detector 34 becomes excessive. Because blood vessels inside the measurement site H deform due to being pressed when the contact pressure is excessive, the measurement of a pulse rate with high accuracy becomes difficult. When the tightening of the mounting unit 12 to the measurement site H is excessively weak, the contact pressure applied to the measurement site H from the detector 34 is insufficient. When the contact pressure is insufficient, not only is the contact pressure low, but also a gap may be formed between the measurement site H and the detector 34, and emitted light from the light emitter 341 or emitted light from the measurement site H may leak to the outside via the gap. Therefore, the measurement of a pulse rate with high accuracy becomes difficult. In the first embodiment, the biometric apparatus 100 evaluates the appropriateness of the mounting state of the detector 34 on the measurement site H in the consideration of the circumstances above.

The controller 31 is an arithmetic processor such as a central processing unit (CPU) or a field-programmable gate array (FPGA), and controls the entirety of the biometric apparatus 100. The storage device 32 is a non-volatile semi-conductor memory, and stores a program executed by the controller 31, and various data used by the controller 31.

FIG. 4 is a block diagram illustrating a functional configuration of the controller 31. As illustrated in FIG. 4, the controller 31 of the first embodiment realizes an analysis unit 41, a calculation unit 42, and an evaluation unit 43 by executing the program stored in the storage device 32. It is possible to adopt a configuration where the functions of the controllers 31 are distributed in a plurality of integrated circuits, or a configuration where part or the entirety of the functions of the controller 31 is realized via a dedicated electronic circuit.

The analysis unit 41 calculates a pulse rate of the measurement site H based on the detection signal D generated by the detector 34. A well-known technology is arbitrarily adopted to the calculation of a pulse rate via the analysis unit 41. The analysis unit 41 sequentially calculates pulse rates at predetermined time intervals. The display device 33 displays the pulse rates sequentially calculated by the analysis unit 41. That is, the display device 33 displays the pulse rates in a chronological order.

The calculation unit 42 calculates an evaluation index E in response to the resistance between the first electrode 21 and the second electrode 22. The evaluation index E is a current value of current flowing between the first electrode 21 and the second electrode 22 when a predetermined voltage is applied between the first electrode 21 and the second electrode 22. If a tensile load is applied to the first portion 121, the resistance between the first electrode 21 and the second electrode 22 increases. Therefore, the greater the tensile load applied to the first portion 121 is, that is, the stronger the tightening of the mounting unit 12 to the measurement site H is, the smaller numerical value the evaluation index E becomes. As understood from the description above, the evaluation index E is an index of the mounting state of the detector 34 on the measurement site H.

The evaluation unit 43 evaluates the mounting state of the detector 34 on the measurement site H in response to the evaluation index E calculated by the calculation unit 42. Specifically, the evaluation unit 43 determines the appropriateness of a contact pressure between the detector 34 and the measurement site H in response to the evaluation index E.

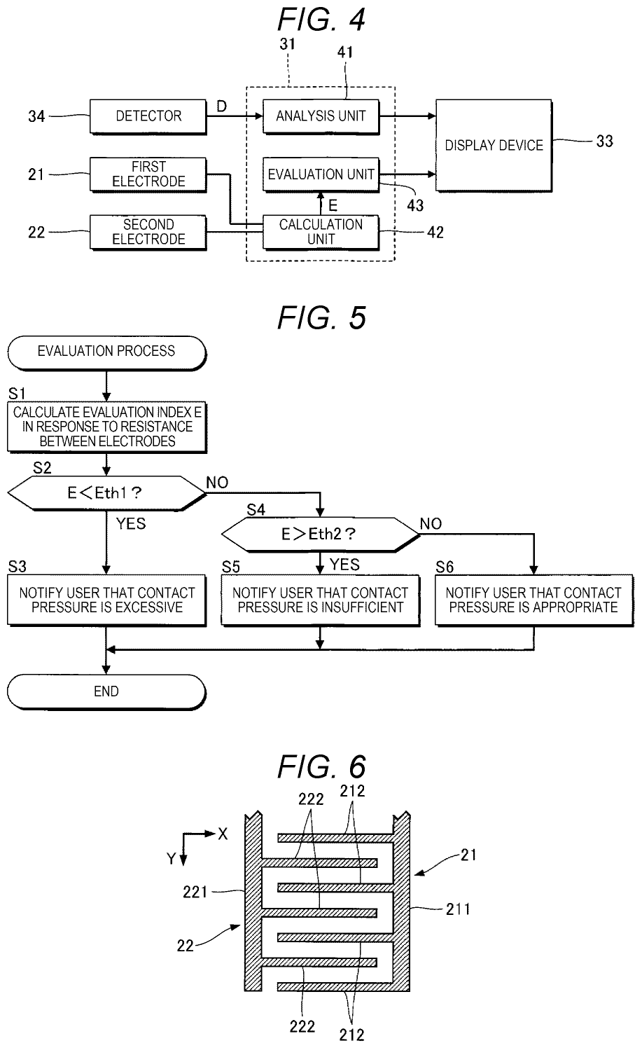

FIG. 5 is a flowchart illustrating a specific sequence of a process (hereinafter, referred to as “evaluation process”) of evaluating the mounting state of the detector 34 on the measurement site H. The evaluation process of FIG. 5 is executed at predetermined time intervals in parallel to pulse rates being calculated by the analysis unit 41. If the evaluation process starts, the calculation unit 42 calculates the evaluation index E in response to the resistance between the first electrode 21 and the second electrode 22 (S1).

The evaluation unit 43 determines whether the evaluation index E calculated by the calculation unit 42 is less than a predetermined threshold value Eth1 (S2). The threshold value Eth1 is statistically or experimentally set such that the evaluation index E is less than the threshold value Eth1 when the contact pressure between the detector 34 and the measurement site H is excessive. When the evaluation index E is less than the threshold value Eth1 (S2: YES), the evaluation unit 43 notifies a user that the contact pressure between the detector 34 and the measurement site H is excessive (S3). The evaluation unit 43 instructs the display device 33 to display a message that the mounting unit 12 has to be less tightened. Upon confirming the message, the user less tightens the mounting unit 12 by appropriately operating the operation knob 125 of the adjustment section 123.

On the other hand, when the evaluation index E is greater than the threshold value Eth1 (S2: NO), the evaluation unit 43 determines whether the evaluation index E calculated by calculation unit 42 is greater than a predetermined threshold value Eth2 (S4). The threshold value Eth2 is a numerical value greater than the threshold value Eth1. The threshold value Eth2 is statistically or experimentally set such that the evaluation index E is greater than the threshold value Eth2 when the contact pressure between the detector 34 and the measurement site H is insufficient. When the evaluation index E is greater than the threshold value Eth2 (S4: YES), the evaluation unit 43 notifies the user that the contact pressure between the detector 34 and the measurement site H is insufficient (S5). The evaluation unit 43 instructs the display device 33 to display a message that the mounting unit 12 has to be strongly tightened. Upon confirming the message, the user tightens the mounting unit 12 by appropriately operating the operation knob 125 of the adjustment section 123.

When the evaluation index E is less than the threshold value Eth2 (S4: NO), the evaluation unit 43 notifies the user that the detector 34 is appropriately mounted on the measurement site H (S6). The evaluation unit 43 instructs the display device 33 to display a message that the mounting unit 12 is appropriately tightened. As illustrated above, the evaluation unit 43 evaluates that the mounting state of the detector 34 is appropriate when the evaluation index E is a numerical value in a range from the threshold value Eth1 to the threshold value Eth2. The evaluation unit 43 evaluates that the mounting state of the detector 34 is inappropriate when the evaluation index E is a numerical value outside the range.

As described above, in the first embodiment, the first electrode 21 and the second electrode 22 are formed on the first portion 121 made of pressure-sensitive conductive resin, and the mounting state of the detector 34 is evaluated based on the evaluation index E in response to the resistance between the first electrode 21 and the second electrode 22. Therefore, there is an advantage that the mounting state of the detector 34 can be appropriately evaluated because the stretching and contraction of the entirety of the mounting unit 12 is additionally evaluated compared to a configuration where the mounting state of the detector 34 is evaluated in response to a local pressure detected by a pressure sensor.

In the first embodiment, because the first electrode 21 and the second electrode 22 are formed on the surface F1 of the first portion 121 of the mounting unit 12, which is on the opposite side of the measurement site H, the first electrode 21 and the second electrode 22 are prevented from coming into contact with the measurement site H. Therefore, it is possible to reduce the possibility that the mounting state of the detector 34 is erroneously evaluated due to the first electrode 21 and the second electrode 22 coming into contact with the measurement site H.

### Second Embodiment

A second embodiment of the present disclosure will be described. In each of the following illustrations, reference signs used in the description of the first embodiment will be assigned to elements having the same functions as in the first embodiment, and the detailed description thereof will be appropriately omitted.

FIG. 6 is a plan view of the first electrode 21 and the second electrode 22 in the second embodiment of the present disclosure. In the second embodiment, the first electrode 21 and the second electrode 22 of FIG. 6 are formed on the surface F1 of the first portion 121 of the mounting unit 12. The Y direction of FIG. 6 is an example of a “first direction”, and the X direction of FIG. 6 is an example of a “second direction”.

As illustrated in FIG. 6, the first electrode 21 includes a first base portion 211 extending in the Y direction, and a plurality of first branch portions 212 extending from the first base portion 211 toward the second electrode 22 along the X direction. The plurality of first branch portions 212 are arranged in the Y direction with gaps therebetween. Similarly, the second electrode 22 includes a second base portion 221 extending in the Y direction, and a plurality of second branch portions 222 extending from the second base portion 221 toward the first electrode 21 along the X direction. The plurality of second branch portions 222 are arranged in the Y direction with gaps therebetween. That is, each of the first electrode 21 and the second electrode 22 has a planar comb tooth-like shape.

As illustrated in FIG. 6, the first branch portions 212 and the second branch portions 222 are alternately arranged in the Y direction. That is, one of the second branch portions 222 is formed between two of the first branch portions 212 which are adjacent to each other in the Y direction. Similar to the first embodiment, the appropriateness of the mounting state of the detector 34 is evaluated based on the evaluation index E in response to the resistance between the first electrode 21 and the second electrode 22.

In the second embodiment, the same advantages as in the first embodiment are realized as well. In the second embodiment, because the first branch portions 212 of the first electrode 21 and the second branch portions 222 of the second electrode 22 are alternately arranged in the Y direction, it is easy to secure a current flow path in the first portion 121 between the first electrode 21 and the second electrode 22. In the aforementioned configuration, even when the stretching and contraction amount of the first portion 121 is small, the resistance between the first electrode 21 and the second electrode 22 changes enough. Therefore, there is an advantage that the mounting state of the detector 34 on the measurement site H can be appropriately evaluated.

### Third Embodiment

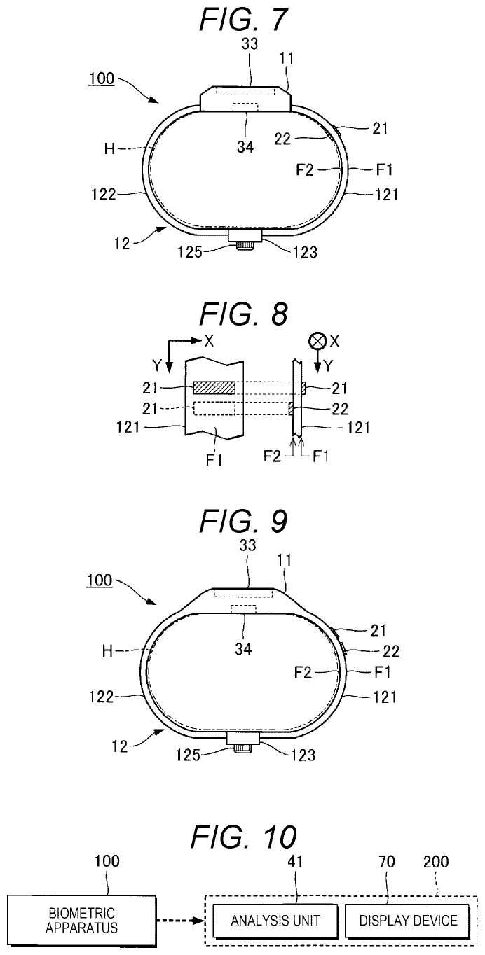

FIG. 7 is a lateral view of the biometric apparatus 100 according to a third embodiment of the present disclosure. As illustrated in FIG. 7, similar to the first embodiment, the first electrode 21 of the third embodiment is formed on the surface F1 of the first portion 121, which is on the opposite side of the measurement site H. In contrast, the second electrode 22 is formed on a surface F2 of the first portion 121, which is on the opposite side of the surface F1. The surface F2 is a surface (that is, inner peripheral surface of the mounting unit 12) of the first portion 121, which faces the measurement site H. The surface F2 corresponds to a specific example of a “second surface”. The first electrode 21 and the second electrode 22 overlap each other in a plan view of the first portion 121. That is, in the third embodiment, the biometric apparatus 100 adopts a structure in which the first portion 121 is interposed between the first electrode 21 and the second electrode 22. Similar to the first embodiment, the calculation unit 42 calculates the evaluation index E in response to the resistance between the first electrode 21 and the second electrode 22.

Similar to the first embodiment, the stronger the tightening of the mounting unit 12 to the measurement site H is, the further a tensile load applied to the first portion 121 increases, and thus the resistance between the first electrode 21 and the second electrode 22 increases. Therefore, the stronger the tightening of the mounting unit 12 to the measurement site H is, the smaller numerical value the evaluation index E becomes. Similar to the first embodiment, the mounting state of the detector 34 is evaluated based on the evaluation index E.

In the third embodiment, the same advantages as in the first embodiment are realized as well. In the third embodiment, because the first electrode 21 and the second electrode 22 are formed opposite to each other with the first portion 121 interposed therebetween, advantageously, it is possible to reduce the possibility that the first electrode 21 and the second electrode 22 are directly and electrically connected to each other.

### Fourth Embodiment

FIG. 8 illustrates a plan view and a lateral view of the first portion 121 in a fourth embodiment of the present disclosure. As illustrated in FIG. 8, in the fourth embodiment, similar to the second embodiment, the first electrode 21 is formed on the surface F1 of the first portion 121, and the second electrode 22 is formed on the surface F2 of the first portion 121. As illustrated in FIG. 8, the first electrode 21 and the second electrode 22 do not overlap each other in a plan view as seen from above in a direction perpendicular to the surface F1 or the surface F2. Similar to the first embodiment, the calculation unit 42 calculates the evaluation index E in response to the resistance between the first electrode 21 and the second electrode 22, and the mounting state of the detector 34 is evaluated based on the evaluation index E.

In the fourth embodiment, the same advantages as in the first embodiment are realized as well. In the fourth embodiment, the resistance between the first electrode 21 and the second electrode 22 changes in response to both stretching and contraction of the first portion 121 in the direction perpendicular to the surface F1 and stretching and contraction in a direction parallel with the surface F1. Therefore, the evaluation index E, in which the mounting state of the detector 34 is appropriately reflected, can be calculated in response to the resistance between the first electrode 21 and the second electrode 22.

FIG. 8 illustrates the configuration where the first electrode 21 and the second electrode 22 do not overlap each other in a plan view, and on the other hand, a configuration where the first electrode 21 and the second electrode 22 partially overlap each other in a plan view may be adopted. As understood from the description above, according to the comprehensive description of the configuration of the fourth embodiment, the first electrode 21 and the second electrode 22 have a region where both do not overlap each other in a plan view.

### Fifth Embodiment

FIG. 9 is a lateral view of the biometric apparatus 100 in a fifth embodiment. As illustrated in FIG. 9, in the fifth embodiment, the housing unit 11, the first portion 121, and the second portion 122 are integrally formed. Specifically, the housing unit 11, the first portion 121, and the second portion 122 are integrally formed by injection molding of pressure-sensitive conductive resin. Similar to the first embodiment, the first electrode 21 and the second electrode 22 are formed on the surface F1 of the first portion 121. The operation of the biometric apparatus 100 also is the same as in the first embodiment.

In the fifth embodiment, the same advantages as in the first embodiment are realized as well. FIG. 9 illustrates the configuration where the first electrode 21 and the second electrode 22 are formed in the same manner as in the first embodiment. On the other hand, the first electrode 21 and the second electrode 22 may be formed in the same manner as in any of the second to fourth embodiments.

### Modification Example

The embodiments illustrated above can be modified in various forms. Specific modification forms will be illustrated below. It is possible to appropriately combine two or more forms arbitrarily selected from the illustration below.

(1) Each of the aforementioned embodiments illustrates the configuration where the mounting unit 12 includes the first portion 121 and the second portion 122; however, the specific configuration of the mounting unit 12 is not limited to the illustration above. A ring-shaped member made of pressure-sensitive conductive resin may be used as the mounting unit 12. The mounting unit 12 where the first portion 121 and the second portion 122 are integrally formed may be installed on the housing unit 11.

(2) In each of the aforementioned embodiments, the entirety of the first portion 121 of the mounting unit 12 is made of pressure-sensitive conductive resin, and on the other hand, a pressure sensing unit made of pressure-sensitive conductive resin may be installed as part of the first portion 121. As understood from the description above, if the mounting unit 12 which mounts the detector 34 on the measurement site H is configured to include a pressure sensing unit that is made of pressure-sensitive conductive resin and can elastically stretch and contract, the position or the shape of the pressure sensing unit is arbitrarily determined.

(3) In each of the aforementioned embodiments, a pulse rate of the measurement site H is measured as biometric information; however, biometric information calculated by the analysis unit 41 is not limited to a pulse rate. An oxygen saturation (SpO2) or a blood component concentration may be measured as biometric information. Examples of the blood component concentration include a blood glucose concentration, a hemoglobin concentration, a blood oxygen concentration, and a triglyceride concentration. A blood flow index at the measurement site H can be measured as biometric information. Examples of the blood flow index include a blood flow velocity, a blood volume index, and a blood flow rate index. The blood volume index is an index (so-called MASS value) indicating the blood volume at the measurement site H, and the blood flow rate index is an index (so-called FLOW value) indicating a blood flow rate at the measurement site H. In the configuration where the blood flow index is measured as biometric information, a light emitting element irradiating the measurement site H with coherent laser light is preferably used as the light emitter 341.

(4) In each of the aforementioned embodiments, the configuration where the biometric apparatus 100 includes the analysis unit 41, and on the other hand, as illustrated in FIG. 10, the analysis unit 41 may be installed in an information apparatus 200 separate from the biometric apparatus 100. The information apparatus 200 is an information device such as a mobile phone, a smart phone, or a tablet PC, and includes the analysis unit 41 and a display device 70. The biometric apparatus 100 transmits the detection signal D to the information apparatus 200 via a wire or wirelessly. The analysis unit 41 of the information apparatus 200 calculates biometric information at the measurement site H by analyzing the detection signal D received from the biometric apparatus 100, and displays the biometric information on the display device 70. The display device 70 of the information apparatus 200 may display the result of evaluating the mounting state of the detector 34 via the evaluation unit 43. As understood from the description above, the analysis unit 41 and the display device 33 can be omitted from the biometric apparatus 100.

(5) In each of the aforementioned embodiments, the display device 33 displays the result of evaluating the mounting state of the detector 34 via the evaluation unit 43; however, a configuration where a user is notified of an evaluation result is not limited to the illustration above. A user may be notified of an evaluation result via voice.

What is claimed is:

1. A biometric apparatus comprising:
a detector generating a detection signal in response to a state of a living body;
a mounting unit mounting the detector to the living body, and including a pressure sensing unit made of pressure-sensitive conductive resin and configured to elastically stretch and contract, and a first electrode and a second electrode formed in the pressure sensing unit;
a calculation unit calculating an evaluation index of a contact pressure in response to a resistance between the first electrode and the second electrode; and
an evaluation unit evaluating a mounting state of the detector in response to the evaluation index.

2. The biometric apparatus according to claim 1, wherein,
when the evaluation index is less than a threshold value of the detector for a measurement site of the living body, the evaluation unit notifies that a contact pressure between the living body and the detector is excessive.

3. The biometric apparatus according to claim 1, wherein,
the first electrode and the second electrode are provided in a surface of the pressure sensing unit, which is on the opposite side of the living body.

4. The biometric apparatus according to claim 1, wherein,
the first electrode includes a first base electrode extending in a first direction, and a plurality of first branch electrodes extending from the first base electrode, which is a starting point, in a second direction intersecting the first direction,
the second electrode includes a second base electrode that extends in the first direction and is provided apart from the first base electrode in the second direction, and a plurality of second branch electrodes that extend from the second base electrode, which is a starting point, in an opposite direction of the second direction, and
the first branch electrodes and the second branch electrodes are alternately arranged in the first direction.

5. The biometric apparatus according to claim 1, wherein,
the first electrode is provided in a first surface of the pressure sensing unit, and
the second electrode is provided in a second surface of the pressure sensing unit, which is on the opposite side of the first surface.

6. The biometric apparatus according to claim 5, wherein,
the first electrode and the second electrode have a region where both do not overlap each other when seen from above in a direction perpendicular to the first surface.
